(19) United States
(12) Patent Application Publication
(10) Pub. No.: US 2020/0085291 A1
(43) Pub. Date: Mar. 19, 2020

(54) Apparatus and a Method of Quantifying Visual Patterns

(71) Applicant: Hyderabad Eye Research Foundation, Hyderabad (IN)

(72) Inventors: Premnandhini Satgunam, Hyderabad (IN); Ashutosh Richhariya, Hyderabad (IN); Gaddam Manoj Kumar, Guntur (IN); Jagadesh Rao Rudrapankte, Howrah (IN); Kabeer Das Mandala, Nalgonda (IN); Ashish Kumar Singh, Varanasi (IN)

(21) Appl. No.: 16/689,602

(22) Filed: Nov. 20, 2019

Related U.S. Application Data
(63) Continuation-in-part of application No. 15/566,618, filed on Oct. 13, 2017, now Pat. No. 10,517,475.

(30) Foreign Application Priority Data
Aug. 19, 2015 (IN) 4341/CHE/2015

(51) Int. Cl. A61B 3/024; G03B 37/04; G06F 3/01; G03B 21/56; A61B 3/00

(52) U.S. Cl. CPC A61B 3/024; G03B 37/04; A61B 3/0008; G06F 3/012; G03B 21/56; G06F 3/013

(57) ABSTRACT

The present disclosure relates to an apparatus and method for quantifying visual fields of a subject. The apparatus includes a dome structure for accommodating at least a part of body of the subject within the dome structure, a projection device mounted on the dome structure configured to project at least one of light stimuli and an image on inner surface of the dome structure and to capture one or more responses of the subject to one of the projected light stimuli and the image. The apparatus further includes a processor configured to analyze the one or more captured responses of the subject to quantify visual fields of the subject.

### CROSS-REFERENCE TO RELATED APPLICATIONS

This application is a Continuation-in-part under 35 U.S.C. § 120 of U.S. application Ser. No. 15/566,618 filed on Oct. 13, 2017, which is a U.S. National Stage filing under 35 U.S.C. § 371 of International Application Serial No. PCT/IB2016/054148, the disclosures of which are hereby incorporated herein by reference in their entirety.

### BACKGROUND

“Visual field” is the extent of peripheral vision of a person while looking straight ahead. A device used to measure the extent or gaps in the visual field is called as ‘perimeter’. The perimeter testing serves as a screening tool to detect diseases of the eye and the visual pathway that connects the eye to the brain. Testing the visual fields is as important in children as it is in adults as there are several diseases that occur in both age groups affecting the visual fields (e.g. glaucoma, hemianopia). It is also known that many children with multiple disabilities (e.g. cerebral palsy) also have visual field defects.

Presently existing perimetric testing requires an individual to be seated with a head and chin firmly placed in the device and to respond to a detection of a moving/flashed light with a button press. This becomes cumbersome for the children to keep pressing the button and in case of infants it is not practical to take readings using said device. Thus, there is need for a novel solution for measuring visual field in infants, children, and adults.

### SUMMARY OF THE INVENTION

The features and advantages realized through the techniques of the present disclosure are brought out. Other embodiments and aspects of the disclosure are described in detail herein and are considered a part of the claimed disclosure.

The shortcomings of the prior art are overcome, and additional advantages are provided through the present disclosure. Additional features and advantages are realized through the techniques of the present disclosure. Other embodiments and aspects of the disclosure are described in detail herein and are considered a part of the claimed disclosure.

In one embodiment, the present disclosure describes an apparatus to quantify visual fields of a subject. The apparatus comprises a dome structure for accommodating at least a part of body of a subject within the dome structure, a projection device mounted on the dome structure configured to project at least one of light stimuli and an image on inner surface of the dome structure and capture one or more responses of the subject to one of the projected light stimuli and the image. The apparatus further comprises a processor configured to analyze the one or more responses of the subject to quantify visual fields of the subject.

In another embodiment, the present disclosure relates to a method of quantifying visual fields of a subject. The method comprises projecting at least one of light stimuli and an image from a projection device on to a dome structure that accommodates at least a part of body of the subject and capturing one or more responses of the subject to one of the projected light stimuli and the image. The method further comprises analyzing the one or more responses of the subject to quantify visual fields of the subject.

It is to be understood that the aspects and embodiments of the invention described above may be used in any combination with each other. Several of the aspects and embodiments may be combined together to form a further embodiment of the invention. The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of device or system and/or methods in accordance with embodiments of the present subject matter are now described, by way of example only, and with reference to the accompanying figures, in which:

FIG. 1 illustrates architecture of a system to quantify visual fields, in accordance with some embodiments of the present disclosure;

FIG. 2A illustrates a dome structure along with an exemplary projection device, in accordance with some embodiments of the present disclosure;

FIG. 2B illustrates a block diagram of the exemplary projection device of FIG. 2A, in accordance with some embodiments of the present disclosure;

FIG. 3A illustrates a dome structure along with another exemplary projection device, in accordance with some embodiments of the present disclosure;

FIG. 3B illustrates a block diagram of the exemplary projection device of FIG. 3A, in accordance with some embodiments of the present disclosure;

FIG. 4A illustrates a dome structure along with yet another exemplary projection device, in accordance with some embodiments of the present disclosure;

FIG. 4B illustrates a block diagram of the exemplary projection device of FIG. 4A, in accordance with some embodiments of the present disclosure; and

FIG. 5 shows a flowchart illustrating a method to quantify visual fields, in accordance with some embodiments of the present disclosure.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a device or system or apparatus proceeded by “comprises . . . a” does not, without more constraints, preclude the existence of other elements or additional elements in the device or system or apparatus.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

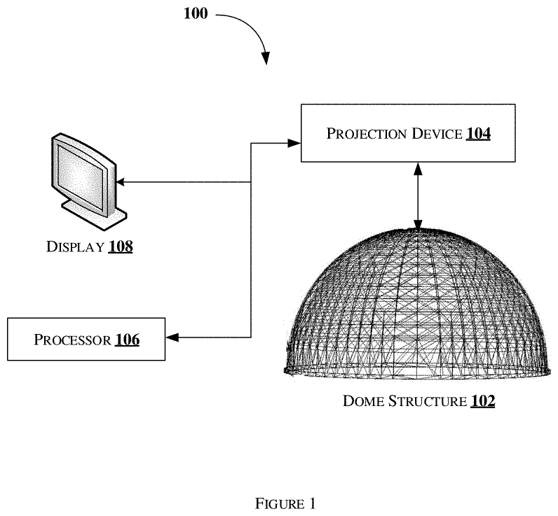

FIG. 1 illustrates an architecture of a system 100 to quantify visual fields in accordance with some embodiments of the present disclosure. The system 100 comprises a dome structure 102, a projection device 104, a processor 106, and a display unit 108. The dome structure 102 is a hemispherical shaped dome having a concave inner surface and is built with one of a steel and plastic skeleton. In one embodiment, diameter of the hemispherical dome is 120 cm, thus allowing an infant to be placed comfortably in a supine position. In another embodiment, diameter of the hemispherical dome can be 60 cm and thus allowing at least head of the subject to be placed comfortably in either one of sitting, sleeping position, or on supine position. The subject herein includes one of infant, children, and adult. In one embodiment, the dome structure 102 is portable. The projection device 104 is mounted on the dome structure 102 to project one of light stimuli and an image on the concave inner surface of the dome structure 102 and to capture one or more responses of the subject for the projected light stimuli and the image. In one embodiment, the projection device 104 is configured to project a single beam of light. In another embodiment, the projection device 104 is configured to project any of animated characters or moving images which looks very attractive for the subject. The projection device 104 includes at least one of an imaging sensor, a fixation light source, and a light source unit that are mounted on the dome structure. In one example, the imaging sensor of the projection device 104 is an infra-red (IR) camera that is configured to capture one or more responses of the subject to the projected light stimuli. The processor 106 is configured to analyse the one or more responses of the subject to quantify visual fields of the subject. In embodiment, one or more responses of the subject includes at least one of head and eye movement of the subject in response to the projected light stimuli.

In one embodiment, the processor 106 may include specialized processing units such as integrated system controllers, memory management control units, floating point units, graphics processing units, digital signal processing units, etc. The display unit 108 is a graphical user interface that is configured to display the one or more responses of the subject captured by the imaging sensor of the projection device 104. In some embodiments, the processor 106 may be disposed in communication with a memory (not shown). The memory may store a collection of data related to intensity and position of light projected from the projection device. The memory also stores previous history of intensity and position of light stimuli projected from the projection device, and the one or more responses captured in response to the projected light stimuli as training data. In some embodiments, the processor 106 is configured to correct or modify the intensity and position of light projected from the projection device based on the previous history of intensity and position of light stimuli as well as the one or more responses captured in response to the projected light data stored in the memory as training data using any one of artificial intelligence (AI) and machine learning (ML) technology.

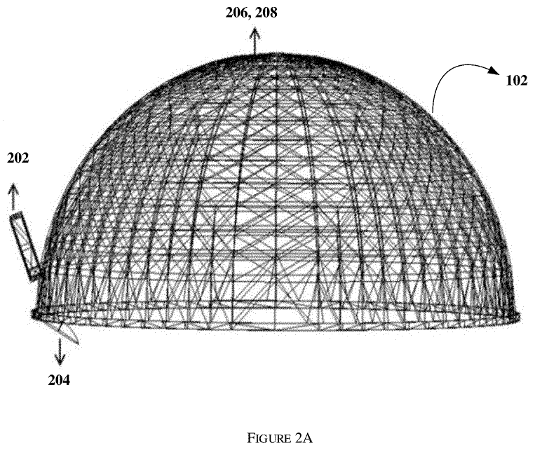

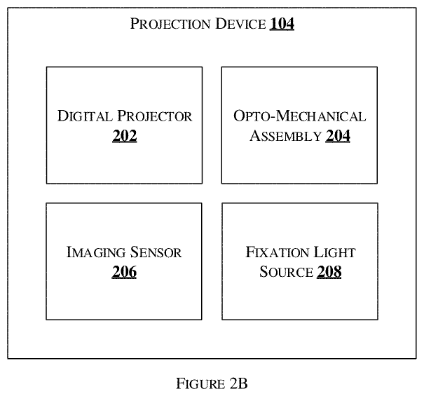

FIG. 2A illustrates a dome structure along with an exemplary projection device, in accordance with some embodiments of the present disclosure. The projection device 104 as illustrated in FIG. 1 is mounted on the dome structure 102 that includes a digital projector 202, an opto-mechanical assembly 204, an imaging sensor 206, and a fixation light source 208. The digital projector 202 is coupled to a power source (not shown) and is configured to emit at least one of light stimuli and the image. The image emitted by the digital projector 202 may be static image or dynamic image, for example, animation image which is more attractive to the subject, thereby enabling quick response from the subject. In one embodiment, the digital projector 202 and the opto-mechanical assembly 204 are mounted at one end of the dome structure 102 as shown in FIG. 2A. The opto-mechanical assembly 204, may for example, include at least a plurality of lens and mirrors mounted on the dome structure using a fastening means. The plurality of lens and mirrors receives the at least one of light stimuli and the image from the digital projector 202 and focuses the received light stimuli and the image onto a concave inner surface of the dome structure 102.

FIG. 2B illustrates a block diagram of the exemplary projection device of FIG. 2A, in accordance with some embodiments of the present disclosure. The projection device 104, as shown in FIG. 2b, includes the digital projector 202, the opto-mechanical assembly 204, the imaging sensor 206, and the fixation light source 208. The digital projector 202 is mounted on an outer surface of the dome structure 102 and is configured to emit at least one of light stimuli and an image. The image emitted by the digital projector 202 may be static image or dynamic image, for example, animation image which is more attractive to the subject than conventional way of emitting a light spot. In one embodiment, the processor 106 controls the location and intensity of the at least one of light stimuli and the image emitted by the digital projector 202. The processor 106 is configured to vary the intensity and location of the at least one of light stimuli and the image based on the previous history of intensity and position of light stimuli and one or more analysed responses of the subject. The opto-mechanical assembly 204 is coupled to the digital projector 202 and is configured to focus the at least one of light stimuli and image emitted by the digital projector 202 onto the concave inner surface of the dome structure 102. The imaging sensor 206 is mounted on top portion of the dome structure 102, such that the imaging sensor is configured to capture one or more responses of the subject to one of the projected light stimuli and the image. In an embodiment, the top portion of the dome structure 102 is provided with an opening/aperture, to receive and couple the imaging sensor 206 with the dome structure 102. The imaging sensor 206 may be for example, an infra-red (IR) camera that is configured to capture the one or more responses of the subject to the projected light stimuli, the captured one or more responses being at least one of head and eye movement of the subject in response to the projected light stimuli. The fixation light source 208 is coupled to the imaging sensor 206 and is configured to emit the light from centre of dome such that the subject looks straight ahead at the centre when there is projection of light stimuli and image.

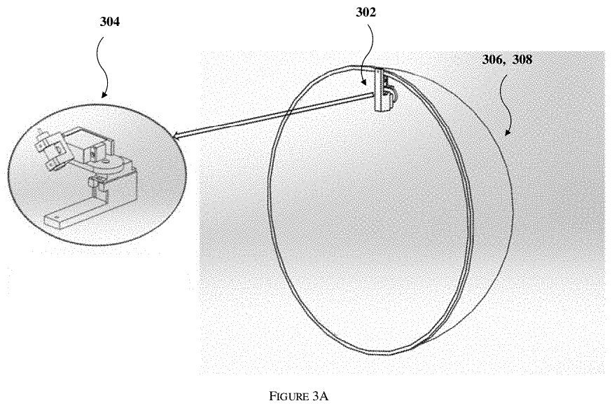

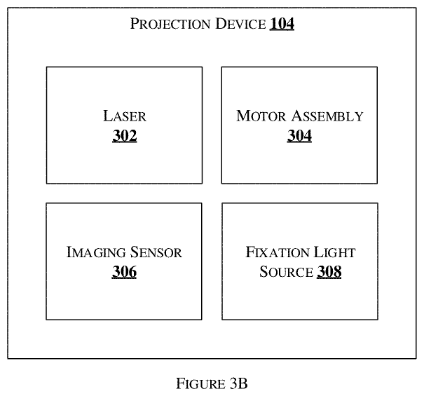

FIG. 3A illustrates a dome structure along with another projection device, in accordance with some embodiments of the present disclosure. The projection device 104 as illustrated in FIG. 1 is mounted on the dome structure 102 that includes a laser 302, a motor assembly 304, an imaging sensor 306, and a fixation light 308. The laser 302 is coupled to a power source (not shown) and is configured to emit at least one of light stimuli and the image. The image emitted by the laser 302 may be static image or dynamic image, for example, animation image which is more attractive to the subject than conventional way of emitting a light spot. In one embodiment, the laser 302 is arranged at one end of the dome structure 102 as shown in FIG. 3A. The motor assembly 304 includes at least a plurality of motors to focus the at least one of light stimuli and the image emitted by the laser power source 302 onto the concave inner surface of the dome structure 102. In one embodiment, the plurality of motors includes a first motor, a second motor, and a third motor coupled to the processor 106. The first motor is configured to rotate the laser 302 in X direction, the second motor is configured to rotate the laser 302 in Y direction, and the third motor is configured to rotate the laser 302 in Z direction. In one embodiment, the plurality of motors may be for example, one or more servomotors that allows for precise control of angular or linear position of the laser 302. In another embodiment, the plurality of motors may be for example, ordinary motors to control angular or linear position of the laser 302.

FIG. 3B illustrates a block diagram of the exemplary projection device of FIG. 3A, in accordance with some embodiments of the present disclosure. The projection device 104, as shown in FIG. 3b, includes the laser 302, the motor assembly 304, the imaging sensor 306, and the fixation light 308. The laser 302 is mounted on an outer surface of the dome structure 102 is configured to emit at least one of light stimuli and an image. In one embodiment, the processor 106 controls the location and intensity of the at least one of light stimuli and the image emitted by the laser 302. The processor 106 varies the intensity and location of the at least one of light stimuli and the image based on the previous history of intensity and position of light stimuli stored in the memory and one or more analysed responses of the subject. The motor assembly 304 is coupled to the laser 302 and is configured to rotate the laser 302 for displaying the at least one of light stimuli and image onto the concave inner surface of the dome structure 102. In one embodiment, the motor assembly 304 comprises a plurality of motors that may include the first motor, the second motor, and the third motor. The first motor is configured to rotate the laser 302 in X direction, the second motor is configured to rotate the laser 302 in Y direction, and the third motor is configured to rotate the laser 302 in Z direction. In one embodiment, the plurality of motors includes one or more servomotors that allows for precise control of angular or linear position of the laser 302. In another embodiment, the plurality of motors includes ordinary motors to control angular or linear position of the laser 302. The processor 106 is configured to vary the speed and movement of one or more of the first, second, and third motors so as to vary the projection location of one of the light stimuli and the image within the dome structure 102. In some embodiments, the processor 106 may be disposed in communication with a memory (not shown). The memory may store a collection of data related to intensity and position of light projected from the laser 302. The memory also stores previous history of intensity and position of light stimuli projected from the laser 302, and the one or more responses captured in response to the projected light stimuli as training data. In some embodiments, the processor 106 is configured to vary the speed and movement of one or more of the first, second, and third motors based on the previous history of intensity and position of light stimuli as well as the one or more responses captured in response to the projected light stored in the memory as training data using any one of artificial intelligence (AI) and machine learning (ML) technology. The imaging sensor 306 is mounted on top portion of the dome structure 102, such that the imaging sensor 306 is configured to capture one or more responses of the subject to one of the projected light stimuli and the image. In an embodiment, the top portion of the dome structure 102 is provided with an opening or aperture, to receive and couple the imaging sensor 306 with the dome structure 102. In one example, the imaging sensor 306 includes an infra-red (IR) camera that is configured to capture the one or more responses of the subject when at least one of head and eye movement of the subject is varied. The fixation light source 308 is coupled to the imaging sensor and is configured to emit the light from centre of dome such that the subject looks straight ahead at the centre when there is no projection of light stimuli and image.

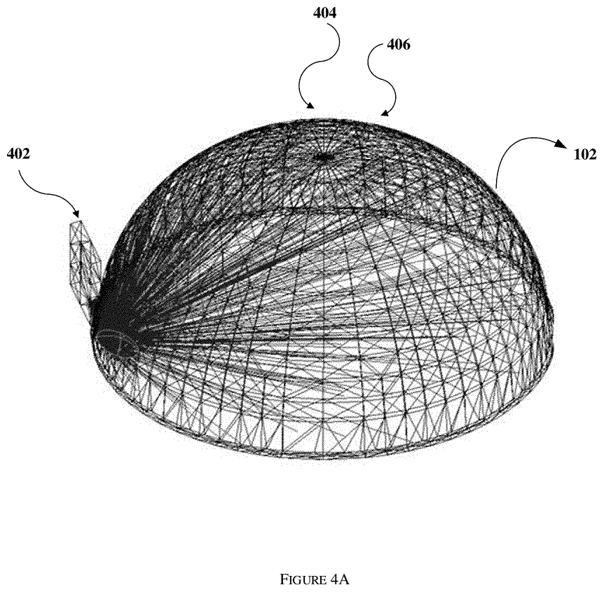

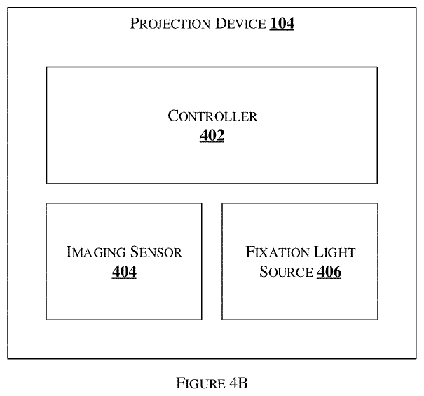

FIG. 4A illustrates a dome structure along with yet another projection device, in accordance with some embodiments of the present disclosure. The projection device 104 as illustrated in FIG. 1 is mounted on the dome structure 102 that includes a controller 402, an imaging sensor 404, and a fixation light source 406. The concave inner surface of the dome structure 102 as shown in FIG. 4a is coated with for example, electroluminescent (EL) paint. The EL paint is a substance that includes a plurality of layers such as an electrically conductive base layer, a dielectric layer, an electroluminescent layer, an electrically conductive clear layer, and a bus bar. The EL paint is configured to emit one of light stimuli and image when electricity is passed through the electrically conductive base layer and the bus bar using a power source (not shown). The controller 402 is configured to control the power source (not shown) to emit the at least one of light stimuli and image on the concave surface of the dome structure coated with the EL paint. The processor 106 is configured to control the controller 402 by varying the intensity of the light stimuli emitted from the EL paint coated within the concave surface of the dome structure 102.

FIG. 4B illustrates a block diagram of the exemplary projection device of FIG. 4B, in accordance with some embodiments of the present disclosure. The projection device, as shown in FIG. 4b, includes the controller 402, the imaging sensor 404, and the fixation light source 406. The controller 402 is mounted on an outer surface of the dome structure 102 and is configured to control a power source (not shown) to emit one of light stimuli and an image on the concave surface of the dome structure 102 coated with the EL paint. The imaging sensor 404 is mounted on top portion of the dome structure 102, such that the imaging sensor 404 is configured to capture one or more responses of the subject to one of the projected light stimuli and the image. In an embodiment, the top portion of the dome structure 102 is provided with an opening or aperture, to receive and couple the imaging sensor 404 with the dome structure 102. In one embodiment, the imaging sensor 404 may be an infra-red (IR) camera that is configured to capture the one or more responses of the subject when at least one of head and eye movement of the subject is varied. The fixation light source 406 is coupled to the imaging sensor 404 and is configured to emit the light from center of dome such that the subject looks at the center when the projected light stimuli and image is not present.

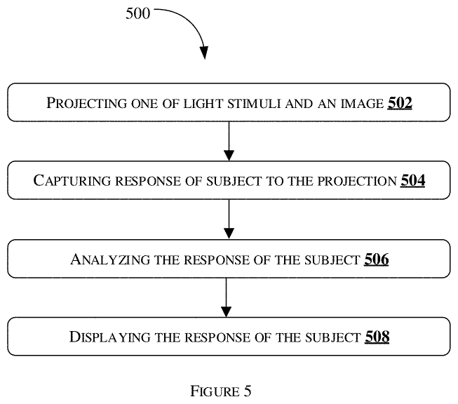

FIG. 5 shows a flowchart illustrating a method of quantify visual fields of a infants, babies with developmental delays, and adults, in accordance with some embodiments of the present disclosure. As illustrated in FIG. 5, the flowchart 500 comprises one or more steps or blocks performed to quantify visual fields of a subject which is in accordance with an embodiment of the present disclosure.

The order in which the method 500 is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

At a “projecting one of light stimuli and an image” block 502, at least one of light stimuli and image is projected from the projection device 104 onto the dome structure 102, wherein the projection device 104 is mounted on the dome structure 102. In one embodiment, the dome structure 102 is a hemispherical shaped dome that has a concave inner surface and is foldable and portable. In one embodiment, the dome structure 102 is built with one of a steel and a plastic skeleton. In one embodiment, diameter of the hemispherical dome 102 is 120 cm, thus allowing an infant to be placed comfortably in a supine position. In another embodiment, diameter of the hemispherical dome 102 can be 60 cm, thus allowing any one of baby and an adult to be placed comfortably in either one of sitting, sleeping position, or on supine position.

In one embodiment, the projection device 104 includes the digital projector 202, the opto-mechanical assembly 204, the imaging sensor 206, and the fixation light source 208. The digital projector 202 is mounted on an outer surface of the dome structure 102 and is configured to emit at least one of light stimuli and an image. The image emitted by the digital projector 202 may be static image or dynamic image, for example, animation image which is more attractive to the subject thereby enabling quick response from the subject. In one embodiment, the processor 106 controls the location and intensity of the at least one of light stimuli and the image emitted by the digital projector 202. The processor 106 is configured to vary the intensity and location of the at least one of light stimuli and the image based on the previous history of intensity and position of light stimuli and one or more analysed responses of the subject. The opto-mechanical assembly 204 is coupled to the digital projector 202 and is configured to focus the at least one of light stimuli and image emitted by the digital projector 202 onto the concave inner surface of the dome structure 102.

In another embodiment, the projection device 104 includes the laser 302, the motor assembly 304, the imaging sensor 306, and the fixation light source 308. The laser 302 is mounted on an outer surface of the dome structure 102 and is configured to emit at least one of light stimuli and an image. In one embodiment, the processor 106 controls the location and intensity of the at least one of light stimuli and the image emitted by the laser power source 302. The processor 106 varies the intensity and location of the at least one of light stimuli and the image based on the previous history of intensity and position of light stimuli stored in the memory and one or more analysed responses of the subject. The motor assembly 304 is coupled to the laser 302 and is configured to rotate the laser for displaying the at least one of light stimuli and image onto the concave inner surface of the dome structure 102. In one embodiment, the motor assembly 304 comprises a plurality of motors that includes a first motor, a second motor, and a third motor. The first motor is configured to rotate the laser 302 is X direction, the second motor is configured to rotate the laser 302 in Y direction, and the third motor is configured to rotate the laser 302 in Z direction. In one embodiment, the plurality of motors includes one or more servomotors that allows for precise control of angular or linear position of the laser power source 302. In another embodiment, the plurality of motors includes ordinary motors to control angular or linear position of the laser power source 302.

In yet another embodiment, the projection device 104 includes the controller 402, the imaging sensor 404, and the fixation light source 406. The controller 402 is mounted on an outer surface of the dome structure 102 and is configured to control one of light stimuli and an image on the concave surface of the dome structure 102 coated with the EL paint.

At a “capturing response of subject upon the projection” block 504, the one or more responses of the subject to a projected light stimuli and image is captured. The projection device 104 includes an imaging sensor mounted on a top outer surface of the dome structure 102 and is configured to capture one or more responses of the subject to a projected light stimuli and image. In one example, the imaging sensor includes an infra-red (IR) camera that is configured to capture the one or more responses of the subject when at least one of head and eye movement of the subject is varied. In one embodiment, capturing one or more response of the subject includes one or more of head and eye movement of the subject.

At a “analyzing the response of the subject” block 506, the one or more responses of the subject to a projected light stimuli and image is analysed to quantify visual fields in the subject. In one embodiment, analyzing the response of the subject includes one of determining gross visual field estimate, determining visual field extent, and determining an actual time taken by an infant or adult to respond to the projected light stimuli and image. In some embodiments, determining gross visual field estimate includes selectively projecting one of a light stimuli and image onto an inner concave surface of the dome structure 102 and capturing one or more response of the subject and the process is terminated if there is no response from the subject to the projected light stimuli and image. In some other embodiments, determining visual field extent includes sequentially projecting one of the light stimuli and image onto the inner surface of dome structure 102 and capturing one or more responses of the subject, populating data points based on the one or more response of the subject to generate visual field isopter. In some embodiments, the actual time taken by the subject to respond is determined based on a time difference between a projected light stimuli or image and one or more responses captured. In one embodiment, one or more response is analysed using a one or more patterns worn on the subject's head for gaze calibration. The one or more patterns include one of a cap, a sticker, and a headband worn on head of the subject.

At a “displaying the response of the subject” block 508, the one or more response of the subject to the projected light stimuli and image is displayed on the display device. In one embodiment, the display device is coupled to the processor can be any one of a cathode ray tube (CRT) or LCD display (or touch screen), for displaying one or more responses of the subject to a user of the perimeter device.

Thus, the above disclosed apparatus enables effective determination of visual fields in infants and patients with special needs. In particular, the apparatus determines visual field estimation based on at least one of head or eye movement detected, thereby accurately determining visual field defects in one or more patients without any manual intervention. Further, the present disclosure provides an automated/corrected intensity and position of the projected light based on previous data without any manual intervention thereby enabling more accurate testing. Such testing would be valuable for infants, children, and adults having neurological conditions for diagnosing, managing, and monitoring the vision problems. Knowing the visual field status of these patients can also enhance the rehabilitation plans for these patients. The device can be easily adapted into pediatric, neurology, and ophthalmology clinics.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

The present disclosure is further described with reference to the following examples, which are only illustrative in nature and should not be construed to limit the scope of the present disclosure in any manner.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

We claim:

1. An apparatus to quantify visual fields of a subject, the apparatus comprising:
a dome structure for accommodating at least a part of body of the subject within the dome structure;
a projection device mounted on the dome structure configured to:
project at least one of light stimuli and an image on inner surface of the dome structure; and
capture one or more responses of the subject to one of the projected light stimuli and the image; and
a processor configured to analyse the one or more responses of the subject to quantify visual fields of the subject.

2. The apparatus according to claim 1, wherein the processor is further configured to vary one of location and intensity of the at least one of light stimuli and image projected by the projection device based on the one or more analyzed responses of the subject.

3. The apparatus according to claim 1, further comprises a display device coupled to the projection device and configured to display the response of the subject captured by the projection device.

4. The apparatus according to claim 1, wherein the dome structure is a hemispherical shaped dome structure and the inner surface of the hemispherical dome shaped structure is concave, wherein the projection device comprises at least one imaging sensor, a fixation light source, and a light source unit mounted on the dome structure.

5. The apparatus according to claim 4, wherein the light source unit includes at least a digital projector capable of emitting at least one of light stimuli and the image, and a plurality of opto-mechanical components configured to focus the at least one of light stimuli and image emitted by the digital projector on to the concave inner surface of the dome structure.

6. The apparatus according to claim 4, wherein the light source unit includes at least a laser and a motor assembly to rotate the laser for displaying the at least one of light stimuli and image on to the concave inner surface of the dome structure.

7. The apparatus according to claim 6, wherein the motor assembly comprises at least a first motor to rotate the laser in X direction and a second motor to rotate the laser in Y direction, wherein the processor is configured to vary the speed and movement of the first and the second motors, based on the captured responses, to vary the location of one of the light stimuli and the image within the dome structure.

8. The apparatus according to claim 1, wherein the concave inner surface of the dome structure is coated with electroluminescent (EL) paint, wherein the projection device includes an imaging sensor, a fixation light source and a controller to control the display of the at least one of light stimuli and image on the concave inner surface of the dome structure coated with the EL paint.

9. The apparatus according to claim 1, wherein the processor is configured to capture the one or more responses of the subject that includes one or more of eye and head movement of the subject.

10. The apparatus according to claim 1, wherein, based on the captured responses, the processor is configured to analyze the response by determining gross visual field estimate, determining visual field extent of the subject, and determining an actual time taken by the subject to respond in response to the projection of at least light stimuli and the image.

11. The apparatus according to claim 1, wherein the processor is configured to analyze the one or more responses using a pattern worn on the subject's head for gaze calibration, wherein the pattern includes at least one of a cap, a sticker, and a headband worn on the head of the subject.

12. A method of quantifying visual fields of a subject, the method comprising:
projecting at least one of light stimuli and an image from a projection device on to a dome structure that accommodates at least a part of body of the subject;
capturing one or more responses of the subject to one of the projected light stimuli and the image; and
analyzing the one or more responses of the subject to quantify visual fields of the subject.

13. The method according to claim 12, further comprising varying at least one of location and intensity of the at least one of light stimuli and image projected by the projection device based on the one or more analyzed responses of the subject.

14. The method according to claim 12, further comprising displaying the response of the subject captured by the projection device on the display device.

15. The method according to claim 12, wherein the dome structure is a hemispherical shaped dome structure and an inner surface of the hemispherical dome shaped structure is concave, wherein the projection device comprises at least one imaging sensor, a fixation light source, and a light source unit mounted on the dome structure.

16. The method according to claim 12, wherein capturing one or more response of the subject includes capturing one or more of eye and head movement of the subject.

17. The method according to claim 12, wherein analyzing the one or more responses includes determining gross visual field estimate, determining visual field extent of the subject, and determining an actual time taken by the subject to respond in response to the projection of at least light stimuli and the image.

18. The method according to claim 12, wherein analyzing the one or more responses comprising analyzing the one or more responses using a pattern worn on the subject's head for gaze calibration, wherein the pattern includes at least one of cap and headband worn on the head of the subject.
